(19) United States
(12) Patent Application Publication
(10) Pub. No.: US 2008/0286315 A1
(43) Pub. Date: Nov. 20, 2008

(54) Methionine Enkephalin as an Adjuvant for Vaccine Immunizations

(75) Inventors: John Huang, San Carlos, CA (US); Ding Chang, San Carlos, CA (US); Shi-lung Lo, North Attleboro, MA (US); Feng Ping Shan, Shenyang (CN)

Correspondence Address: PETERS VERNY , L.L.P., 425 SHERMAN AVENUE, SUITE 230, PALO ALTO, CA, 94306, US

(73) Assignee: Penta Biotech, Inc., Union City, CA (US)

(21) Appl. No.: 12/022,854

(22) Filed: Jan. 30, 2008

Related U.S. Application Data
(60) Provisional application No. 60/898,937, filed on Jan. 31, 2007.

(51) Int. Cl. A61K 39/00; A61K 35/12; A61P 37/00; C12N 5/06

(52) U.S. Cl. 424/277.1; 424/93.7; 435/375; 424/184.1

(57) ABSTRACT

The present invention provides a method of stimulating dendritic cells, comprising contacting the dendritic cells with methionine enkephalin. The present invention also provides methods of enhancing an immune response to an antigen in a mammal. In a first method, dendritic cells are isolated from the mammal. The dendritic cells are then contacted with methionine enkephalin for a time sufficient to stimulate the cells. Next, the stimulated dendritic cells are contacted with the antigen of interest for a time sufficient for the cells to process the antigen. The dendritic cells are then injected into the mammal. The dendritic cells may be injected into the animal either on their own, or along with methionine enkephalin and/or the antigen. In a second method, the antigen of interest and methionine enkephalin are administered to the mammal. The present invention also provides compositions for administration to a mammalian subject having a tumor.

### CROSS-REFERENCE TO RELATED APPLICATIONS

This application claims priority from U.S. Provisional Patent Application No. 60/898,937 filed on Jan. 31, 2007, which is hereby incorporated by reference in its entirety.

### STATEMENT OF GOVERNMENTAL SUPPORT

This invention was not made with government support.

### REFERENCE TO SEQUENCE LISTING, COMPUTER PROGRAM, OR COMPACT DISK

None.

### BACKGROUND OF THE INVENTION

1. Field of the Invention

The present invention relates to the field of immunization. In particular, the present invention relates to the use of methionine enkephalin (MEK) as an adjuvant in vaccine immunizations of humans or animals and to methods of using it in or in combination with vaccines.

2. Related Art

Presented below is background information on certain aspects of the present invention as they may relate to technical features referred to in the detailed description, but not necessarily described in detail. The discussion below should not be construed as an admission as to the relevance of the information to the claimed invention or the prior art effect of the material described.

At present, vaccine immunization is still the best method for preventing infection from microorganisms such as viruses and bacteria in humans and animals. As the field of immunology advances, vaccines are also used in therapy not only for infectious diseases but also for other illnesses such as cancers. Therefore the so-called therapeutic vaccine has been developed. Antigens are the most important element for an effective vaccine. Antigens may be synthesized, for example using recombinant DNA technology, or they may be recovered from an individual. In either case the vaccine may not possess sufficient immunogenicity to facilitate a good immune response, such that an immuno-protection can be established to defend against subsequent invasion of the prospective pathogenic agent. In addition, among normal human populations, there are about 5 to 10% who do not response to a certain vaccine. This may be due to genetic factors, which regulate immune behavior with respect to the specific antigen in the vaccine. This phenomenon of non-response can be alleviated with use of an adjuvant.

Dendritic cells play an important role in the immune system, in that they process antigens and present the MHC-antigen complex to responsive T and B lymphocytes, resulting in activation and proliferation of these lymphocytes. In addition, they are critical for induction of T cell responses resulting in cell-mediated immunity. Accordingly, techniques such as use of recombinant vaccines that target dendritic cells, activation of dendritic cells in vivo, and injection of dendritic cells that have processed antigen in vitro have all been used in an attempt to increase response to antigen in the context of a vaccine. However, there remains a need in the art for processes using dendritic cells and additional factors that may activate or stimulate dendritic cells, either in vitro, in vivo, or both.

Enkephalins are endogenous, opiate-like peptides that are derived from larger peptides, called endorphins. Enkephalins have been found to be one of the ligands for brain morphine receptors. As such, they exhibit antidepressant, anti-anxiety and anticonvulsant activities. In 1979 Wybran et al. reported that normal T lymphocytes possess opiate receptors on human phagocytic leukocytes. These findings have prompted several workers to investigate whether enkephalins possess any potential immunomodulatory activity, For example, in 1982 Plotnikoff et al. discovered that the enkephalins and endorphins had a stimulatory effect on lymphocyte blastogenesis in mice. Subsequent studies from many laboratories have shown that methionine enkephalin (1) augments formation of interleukin 6 by cytokine-stimulated murine macrophages; (2) activates receptors for IL-2, OKT10, and active sheep T red blood cell receptors; (3) augments TNFα production, NK cell activity, and IL-12 p35 mRNA expression; (4) facilitates IL-1, IL-2 and IL-6 production in lymphocytes; and (5) activates or augments inflammatory reactions. However, an association between enkephalins and dendritic cells has yet to be elucidated. As such, there is a need in the art to determine whether enkephalins have an effect on dendritic cells as if such a connection exists, enkephalins may have important adjuvant properties.

### SPECIFIC PATENTS AND PUBLICATIONS

U.S. Pat. No. 4,537,878 to Plotnikoff, issued Aug. 27, 1985, entitled “Process for using endogenous enkephalins and endorphins to stimulate the immune system” discloses the use of enkephalins to stimulate the immune system. It was suggested that enkephalins activate T cells to release interleukin II, which in turn activates the release of interferons and interleukin I and III, thus promoting a cascade of immunological effects.

U.S. Pat. No. 6,136,780 to Zagon, et al., issued Oct. 24, 2000, entitled “Control of cancer growth through the interaction of [Met⁵]-enkephalin and the zeta (ζ) receptor,” describes the use of naltrexone, naloxone and the pentapeptide growth factor [Met⁵]-enkephalin to inhibit and arrest the growth of cancer, including particularly gastrointestinal cancer.

### BRIEF SUMMARY OF THE INVENTION

The following brief summary is not intended to include all features and aspects of the present invention, nor does it imply that the invention must include all features and aspects discussed in this summary.

We have found that methionine enkephalin has a strong effect on the proliferation and maturation of dendritic cells. Thus, in one embodiment, the present invention provides a method of stimulating dendritic cells, comprising contacting the dendritic cells with methionine enkephalin. Stimulation of dendritic cells may include, e.g., increasing expression of the cell surface marker CD 11_{c}, increasing secretion of IL-12, and increasing proliferation of the dendritic cells.

In another embodiment, the present invention provides a method of enhancing an immune response to an antigen in a mammal. In one aspect of this embodiment, dendritic cells are isolated from the mammal. The dendritic cells are then contacted with methionine enkephalin for a time sufficient to stimulate the cells. Next, the stimulated dendritic cells are contacted with the antigen of interest for a time sufficient for the cells to process the antigen. The dendritic cells are then injected into the mammal. The dendritic cells may be injected into the animal either on their own, or along with methionine enkephalin and/or the antigen.

The dendritic cells may be isolated from any tissue of the mammal, but they are preferably isolated from blood. In particular, the dendritic cells may be isolated from a white blood cell enriched fraction of blood. The antigen may also be from any source, including but not limited to a viral antigen, a bacterial antigen, or a tumor antigen, but is preferably a tumor antigen. In this case, the inventive method preferably results in a decrease in the size of a tumor in the mammal compared to injecting dendritic cells that have not been contacted with methionine enkephalin. In addition, the inventive method preferably results in an increase in activity of cytotoxic T lymphocytes compared to injecting dendritic cells not contacted with methionine enkephalin.

In another aspect of this embodiment, the antigen of interest and methionine enkephalin are administered to the mammal. In this aspect, the methionine enkephalin may be administered prior to, along with, or after administration of the antigen. The methionine enkephalin may be administered using any means, including but not limited to intravenously, intra-arterially, intramuscularly, orally, trans-dermally, parentally, via an inhalation spray, via a nasal drop, via an eye-drop, or via a tablet. The antigen may be from any source, including but not limited to a viral antigen, a bacterial antigen, or a tumor antigen, but is preferably a tumor antigen.

In yet another embodiment, the present invention provides a composition for administration to a mammalian subject having a tumor. In one aspect of this embodiment, the composition includes an antigen from the tumor as well as dendritic cells stimulated by methionine enkephalin, where the dendritic cells are obtained from the subject. In another aspect of this embodiment, the composition includes an antigen from the tumor as well as methionine enkephalin.

### BRIEF DESCRIPTION OF THE DRAWINGS

FIG. 1 is a micrograph showing dendritic cells that have been stimulated by IL-4 and GM-CSF.

FIG. 2 is a micrograph showing dendritic cells that have been stimulated by IL-4, GM-CSF, and methionine enkephalin.

### DETAILED DESCRIPTION OF THE PREFERRED EMBODIMENT

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of the clarity, following terms are defined below.

The term “dendritic cells” is used herein to refer to a type of antigen presenting cell, and refers to all subtypes of dendritic cells including, for example, CD8⁺ and CD8⁻ dendritic cells, DC1 and DC2 dendritic cells, and myeloid and lymphoid dendritic cells. In addition the term includes both immature and mature dendritic cells. “Dendritic cells” are further defined in Anjuère, et al., Blood, Vol. 93 No. 2 (Jan. 15), 1999: pp. 590-598, “Definition of Dendritic Cell Subpopulations Present in the Spleen, Peyer's Patches, Lymph Nodes, and Skin of the Mouse.”

Thus, “Mature dendritic cells” are those which have some or all of the following characteristics: numerous processes (veils, dendrites) in their shape; active process formation and movement; antigen capture through a macrophage mannose receptor, DEC-205 receptor; antigen presentation facilitated by high MHC class I and II expression; an abundance of molecules for T cell binding and costimulation, (e.g. CD40, CD54/ICAM-1, CD58/LFA-3, CD80/B7-1 and CD86/B7-2); abundant IL-12 production; resistance to IL-10 DC-restricted molecules: p55, CD83, S 100b; and absence of macrophage-restricted molecules and function: CD14, CD115/c-fms/M-CSF responsiveness, low CD68, myeloperoxidase and lysozyme, bulk endocytic activity (pinocytosis, phagocytosis); and no reversion/conversion to macrophages/lymphocytes.

The term “stimulate” is used to mean activation of maturation, proliferation, or both. In the case of dendritic cells (DCs), these are derived from bone marrow progenitors and circulate in the blood as immature precursors prior to migration into peripheral tissues. Within different tissues, DCs are stimulated to differentiate and become active in the taking up and processing of antigens (Ags), and their subsequent presentation on the cell surface linked to major histocompatibility (MHC) molecules. Upon appropriate further stimulation, DCs undergo further maturation and migrate to secondary lymphoid tissues where they present Ag to T cells and induce an immune response.

The term “methionine enkephalin” is used to mean a pentapeptide with the sequence as set forth in U.S. Pat. No. 4,148,786 to Sarantakis, issued Apr. 10, 1979, entitled “Analgesic polypeptide.” The term “enkephalin analogues” is as described in U.S. Pat. No. 4,304,715 to Hudson, et al., issued Dec. 8, 1981, entitled “Enkephalin analogues.”

A specific structure is illustrated below:

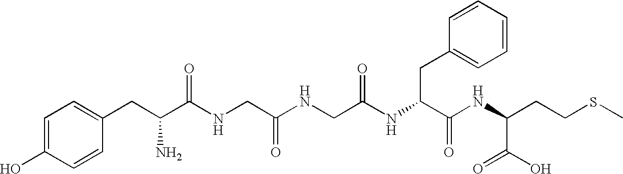

The term “tumor antigen” is used to mean an antigen found in a tumor, which is capable of eliciting an immune response in the host bearing the tumor. Tumor antigens are used in tumor (cancer) vaccines.

Other tumor antigens, for example, are described in M Hareuveni et al., “Vaccination Against Tumor Cells Expressing Breast Cancer Epithelial Tumor Antigen,” Proceedings of the National Academy of Sciences, Vol 87, 9498-9502, which discloses that ninety-one percent of breast tumors aberrantly express an epithelial tumor antigen (ETA) identified by monoclonal antibody H23. Vaccinia recombinants expressing this antigen prevented tumor development.

Cell surface antigens are also included, as described in K. L. Carraway, N. Fregien, K. L. Carraway, and C. A. Carraway, “Tumor sialomucin complexes as tumor antigens and modulators of cellular interactions and proliferation, J. Cell Sci., Oct. 1, 1992; 103(2): 299-307.

A particular tumor antigen may be isolated from a patient's own tumor (patient specific). As described below, the Lewis Lung cancer cell line (LL2 or 3LL), derived from CL57 B1 mouse may serve as a tumor antigen source for animal studies. Lewis Lung cancer cells are known to express the heat shock protein Gp96. See, N Shinagawa, et al., “Immunotherapy with dendritic cells pulsed with tumor-derived gp96 against murine lung cancer is effective through immune response of CD8(+) cytotoxic T lymphocytes and natural killer cells,” Cancer Immunol. Immunother., Feb. 1, 2008; 57(2): 165-74.

### Generalized Method and Compositions

The present invention provides methods and compositions for enhancing an immune response to an antigen in a mammal. The compositions and methods are based on our discovery that methionine enkephalin (MEK) has a strong ability to stimulate the maturation and proliferation of dendritic cells (DCs). As such, MEK may be useful, for example, as an adjuvant in vaccines.

Accordingly, in one embodiment of the present invention, the invention provides methods of enhancing an immune response in an animal. A first method takes advantage of the effect of MEK on DCs by exposing DCs to MEK prior to injecting them into the animal. In a first step of this method, DCs are isolated from the mammal. The DCs may be isolated from a variety of tissues, including but not limited to peripheral blood, bone marrow, and pleural and peritoneal effusions. In a preferred embodiment, blood cells are enriched for white blood cells prior to isolation of the DCs. The DCs may be isolated using any means known in the art. (See, for example, U.S. Pat. No. 6,194,204, issued to Crawford et al., and U.S. Pat. No. 6,491,918, issued to Thomas et al.).

Preferably, the DCs are cultured after isolation. DCs may be cultured using any means known in the art. Typically, they are cultured in a 5% CO₂ incubator at 37° C. In addition the cells are generally cultured with medium containing 10% fetal calf serum (FCS), 10 mg/ml interleukin 4 (IL-4), and 200 ng/ml granulocyte macrophage colony stimulating factor (GM-CSF).

In a second step, DCs are contacted with MEK for a time sufficient to stimulate the dendritic cells. The DCs may be contacted with MEK by any means known in the art. For example, MEK may be added to the dendritic cell culture medium. MEK is added at a concentration and time sufficient to stimulate the maturation, proliferation, or both of the dendritic cells. Preferably, this concentration is between about 60 ng/ml and about 120 ng/ml. More preferably, the concentration of MEK is about 60 ng/ml. Also preferably, the DCs are contacted with MEK for at least a week.

In the next step, the isolated, cultured dendritic cells are contacted with antigen for a time sufficient for the dendritic cells to process the antigen. This period of time is typically at least about two days.

Finally, dendritic cells that have been contacted with MEK and antigen are injected into the mammal. Typically, at least about 10⁶ cells are injected into the animal. In addition, the DCs are preferably injected more than once. Typically, the DCs are injected once a week for six weeks. The DCs may be injected on their own, with antigen, with MEK, or with both antigen and MEK.

Preparation of a suitable preparation of dendritic cells may be carried out as adapted from Morse et al., “Migration of Human Dendritic Cells after Injection in Patients with Metastatic Malignancies,” Cancer Research 59, 56-58, Jan. 1, 1999. Patients may receive 100×10⁶ MEK+antigen stimulated DCs in 15-30 ml of normal saline as an i.v. bolus over 1 mm.

Any antigen may be used to practice the method of the present invention. Examples include bacterial antigens, viral antigens, and tumor antigens. Antigens may be prepared using methods standard in the art. In a preferred aspect of this embodiment, the antigen is a tumor antigen. The antigen may be obtained directly from the tumor, or it may be an antigen known to be associated with a given type of tumor. In the case where the antigen is a tumor antigen, the method of the present invention preferably results in a decrease in the size of the tumor in the animal when compared with animals injected with DCs that have not been exposed to MEK. Regardless of the type of antigen, the inventive method preferably results in an increase in the activity of cytotoxic T lymphocytes (CTLs) compared to animals injected with DCs that have not been exposed to MEK.

The second method of the present invention takes advantage of the potential ability of MEK to stimulate DCs in vivo. According to this method, both MEK and an antigen are administered to an animal. The MEK may be administered prior to, with, or after administration of the antigen. For example, a mixture of MEK and antigen may be injected into the animal. MEK may also be administered, for example, intravenously, intra-arterially, intramuscularly, orally, trans-dermally, parentally, via an inhalation spray, via a nasal drop, via an eye-drop, or via a tablet. As with the first method, any antigen may be used. Preferably, the antigen is a tumor antigen.

The present invention also provides compositions for administration to a mammalian subject having a tumor. In one embodiment, the composition includes an antigen associated with the tumor as well as dendritic cells that have been stimulated by methionine enkephalin. The dendritic cells may be stimulated, e.g., using the methods described above. The antigen may be isolated from the tumor itself, from the bloodstream, or made synthetically. The dendritic cells are preferably isolated from the subject, to prevent rejection of the cells. The amount of antigen needed to elicit an immune response may be determined by routine experimentation using methods known in the art. The number of dendritic cells is preferably at least 10⁶.

In another embodiment, the composition contains an antigen associated with the tumor and methionine enkephalin. Again, the antigen may be provided using any means known in the art. The concentration of antigen may be determined experimentally using techniques standard in the art. The concentration of methionine enkephalin in the composition is preferably between about 60 ng/ml and about 120 ng/ml, more preferably about 60 ng/ml.

The inventive compositions may be used to treat tumors in any mammalian subject, including but not limited to domestic animals and humans.

In yet another embodiment, the present invention provides a method of stimulating dendritic cells with methionine enkephalin. According to this method, dendritic cells are contacted with MEK. This may be accomplished, for example, by adding MEK to a culture of DCs, or by administering MEK to a subject. MEK preferably increases proliferation, maturation or both of DCs. Specifically, MEK preferably increases surface expression of the marker CD11_{c}, secretion of the cytokine IL-12, or both. Preferably, between about 60 ng/ml and about 120 ng/ml of MEK is used to stimulate the DCs, more preferably about 60 ng/ml.

### EXAMPLES

### Example 1

### Effect of Methionine Enkephalin on Maturation and Proliferation of Dendritic Cells

Immature dendritic cells were isolated from spleens of IRM-2 mice with lymphocyte separation solution. The method used was adapted from a method of isolating lymphocytes using a centrifugation technique. First, diluted defibrinated blood was layered on a solution of sodium metrizoate and Dextran or Ficoll® and centrifuged at low speeds for 30 minutes. Differential migration following centrifugation resulted in the formation of several cell layers. Mononuclear cells (lymphocytes and monocytes) and platelets are contained in the banded plasma-lymphocyte separation medium interphase due to their density. Erythrocytes and granulocytes migrate through the gradient and form a pellet to the bottom of the tube. Lymphocytes were recovered by aspirating the plasma layer and removing the cells. After proper washing and treatment, cells were suspended in RPMI-1640 containing 10% FCS and incubated in a CO₂-incubator (37° C., 5% CO₂) for 2 hours before use.

The immature dendritic cells were divided into 6 groups and were centrifuged to remove the supernatants. RPMI-1640 containing 10 ng/ml IL-4, 100 ng/ml GM-CSF, 10% FCS, and MEK at either 0 ng/ml, 7.5 ng/ml, 15 ng/ml, 30 ng/ml, 60 ng/ml or 120 ng/ml was then added to the cells. The final cell density was 2×10⁵ cells/ml. Cells were returned to the CO₂-incubator and the media were replaced in half volumes every other day starting on the 3^{rd} day. Cells were allowed to grow for 9 days and thereafter cell numbers were counted with an inverted phase-contrasted microscope. The group of cells having the highest density was regarded as having the most optimum conditions in terms of concentration for cytokines. Cell density was determined using the MTT [3-(4,5-Dimethylthiazol-2-yl)-2,5-Diphenyltetrazolium Bromide] assay, using methods known in the art. The results of effects on dendritic cell density at different MEK concentrations are shown in Table 1. MEK facilitated the growth and multiplication of dendritic cells in a concentration dependent fashion. The effects were greatest with a MEK concentration at around 60 ng/ml and then decreased thereafter as MEK concentration increased.

TABLE 1
Dendritic Cell Densities and Proliferation
with Different Concentrations of MEK

| | MEK Concentration | Cell Density | |
|---|---|---|---|
| Group | (ng/ml) | (10⁵/ml) | Increase Percent (%) |
| Control | 0 | 2 | 100.0 |
| Exp. 1 | 7.5 | 2.2 ± 0.1 | 127.1 |
| Exp. 2 | 15 | 2.5 ± 0.2 | 133.3 |
| Exp. 3 | 30 | 2.9 ± 1.2 | 159.2 |
| Exp. 4 | 60 | 4.8 ± 0.2 | 246.5 |
| Exp. 5 | 120 | 3.1 ± 0.1 | 165.5 |

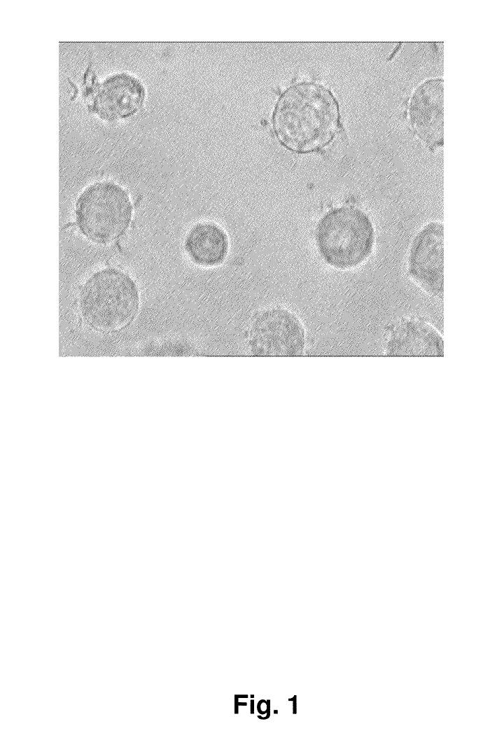

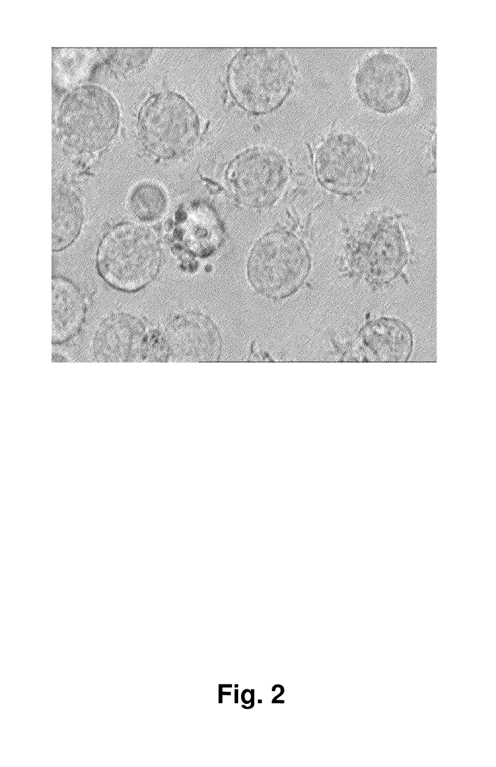

FIGS. 1 and 2 show micrographs of dendritic cells magnified 400×. Cells were isolated and cultured as described above. FIG. 1 shows dendritic cells not treated with MEK. FIG. 2 shows a micrograph of dendritic cells treated with 60 ng/ml MEK. It can be seen from the figures that the addition of MEK leads to an increase in cell density as well as an increase in cell processes (veils or dendrites) on the dendritic cells.

Cells isolated and cultured as described above were processed on the 9^{th} day with the addition of FITC labeled antibodies to CD11_{c} and CD80. CD11_{c} is the most specific cell surface marker for mature dendritic cells. CD80 is another marker that is expressed on mature dendritic cells. Stained cells were analyzed with a flow cell cytometer and cells expressing fluorescence markers were regarded as positive. The positive rates were analyzed and calculated using Bios Consort 30 software. Results of this assay are shown in Table 2. Table 2 shows that the percentage of cells expressing mature dendritic cell-specific markers increases with increasing concentrations of MEK until 60 ng/ml, above which expression decreases.

TABLE 2
Dendritic Cell Surface Marker Expression

| Group | MEK Concentration (ng/ml) | CD11_{C(+)} % | CD80₍₊₎ % |
|---|---|---|---|
| Control | 0 | 47.2 | 50.3 |
| Exp. 1 | 7.5 | 49.9 | 51.3 |
| Exp. 2 | 15 | 51.3 | 54.1 |
| Exp. 3 | 30 | 55.6 | 58.7 |
| Exp. 4 | 60 | 62.7 | 64.6 |
| Exp. 5 | 120 | 57.3 | 63.3 |

To further characterize the cultured cells, cell culture supernatants were collected on the 9^{th} day of incubation and were assayed according to the procedures defined in the IL-12 ELISA Assay Kit obtained from eBioscience, USA. IL-12 is a cytokine known to be secreted by mature dendritic cells. Results of this assay are shown in Table 3. Table 3 shows that the percentage of cells secreting IL-12 increases with increasing concentrations of MEK until 60 ng/ml, above which secretion decreases.

TABLE 3
IL-12 Levels in Supernatants of Cultured Dendritic Cells

| Group | MEK Concentration (ng/ml) | IL-12 Level (pg/ml) |
|---|---|---|
| Control | 0 | 68 |
| Exp. 1 | 7.5 | 102 ± 1.1 |
| Exp. 2 | 15 | 126 ± 0.4 |
| Exp. 3 | 30 | 175 ± 1.6 |
| Exp. 4 | 60 | 256 ± 0.3 |
| Exp. 5 | 120 | 227 ± 1.9 |

### Example 2

### Studies on the Role of Methionine Enkephalin as an Adjuvant in Dendritic Cell Vaccines Against Lewis Lung Carcinoma in Mice

Preparation of Murine Dendritic Cells: Murine dendritic cells were isolated from spleens of IRM-2 mice with lymphocyte separation solution using methods known in the art. After proper washing and treatment, cells were suspended in RPMI-1640 containing 10% FCS and incubated in a CO₂-incubator (37° C., 5% CO₂) for 2 hours to allow cells to attach to the surface of the flask. The medium was then replaced with fresh medium containing 10 mg/ml IL-4 and 200 ng/ml GM-CSF. Cells were then cultured in a 5% CO₂-incubator at 37° C. for 3 days. These cells were ready to be used for dendritic cell culture.

Preparation of Tumor Antigen from Lewis Lung Cancer Cells: Lewis lung cells obtained from the Animal Research Department, Peking Union Medical College, Beijing, China and grown under standard conditions were harvested and cell density was adjusted to 10⁷ cells/ml. Cells were centrifuged at 2,000 RPM for 5 min and re-suspended in saline. Cells were broken by three freeze and thaw cycles and the mixture was centrifuged at 15,000 RPM for 30 min to obtain the soluble (cell-free) portion in the supernatant. This portion was diluted 10× with saline in Eppendorf tubes for further application

Preparation of the Control Dendritic Cell Vaccine: Dendritic cells prepared as described above were cultured in a 5% CO₂-incubator at 37° C. and the medium was replaced every other day. On the 7^{th} day, 100 μl of antigen prepared as described above was added to the cells. On day 9, detached dendritic cells were collected and washed with sterile saline 3 times (with centrifugation at 2,000 RPM, 20 min) and finally resuspended in saline at a concentration of 10⁷ cells/ml. These dendritic cells were designated as DC-vaccine.

Preparation of Methionine Enkephalin Treated Dendritic Cell Vaccine: Dendritic cells prepared as described above were added to medium containing 60 ng/ml MEK after 2 hrs of attachment period. Thereafter, the medium was renewed every 48 hrs. The dendritic cells continued to be cultured and on the 7^{th} day, the dendritic cells were harvested. 100 μl of Lewis lung cell antigen prepared as described above was then added to the dendritic cells. Cells were cultured for another 48 hrs before they were harvested and their cell concentration was adjusted to 10⁷ cells/ml. These dendritic cells were designated as MEK-DC vaccine.

Specific Cytotoxic T Lymphocyte (CTL) Activity Assay: The assay was based on the LDH (lactate dehydrogenase) method. According to this method, a stable cytosolic enzyme, LDH, is released into the cell culture supernatant upon damage of the cytoplasmic membrane due to cytotoxic T lymphocyte attack. Released LDH in culture supernatants is measured with an enzyme reaction of LDH, which oxidizes lactate to pyruvate. Pyruvate in turn reacts with tetrazolium salt INT to form formazan. Formazan dye is measured by absorbance at 490 nm using a spectrophotometer (ELISA reader). The increase in the amount of formazan produced in the cell culture supernatant directly correlates to the increase in numbers of cells lysed by CTL.

The LDH assay measures either apoptosis or necrosis, and kits are available, e.g. from Cayman Chemical and others.

IRM-2 mice were divided into a DC Group and a MEK-DC Group, with 10 mice in each group. For both groups, DC vaccines were injected into the back of each mouse with 2×10⁶ cells, once per week. One week after the 2^{nd} vaccination, the mice were killed. Spleen cells prepared by conventional procedures were used as controls and L₃₋₈ cells (Lewis lung cancer cells) were used for the targeted cells. The targeted cells were distributed at 6×10⁶ cells/well and ratios were set to be 100:1, 50:1, and 25:1. Each assay was repeated in triplicate and activities of CTLs were assayed. The cytotoxicity in killing was calculated as follows:

Killing Rate=(Experimental Group A Value−Effected Cell A Value−Targeted Cell Control A Value)/(Targeted Cell A Value−Target Cell Control A Value)×100%.

CTLs from mice that were vaccinated with MEK activated DCs had significantly higher specific killing rates of Lewis lung cancer cells than CTLs from mice that were vaccinated by the DC-vaccine, as shown in Table 4 (statistically significant where p<0.05).

TABLE 4
CTL Activities toward L₃₋₈ Cells with Two Vaccines

| Effective Target Ratio | 100:1 | 50:1 | 25:1 |
|---|---|---|---|
| DC Vaccine | 41.6% | 35.8% | 26.9% |
| MEK-DC Vaccine | 65.3% | 43.6% | 31.5% |

### Example 3

### Improved Tumor Size Reduction in Animals Injected with MEK-Stimulated Dendritic Cells

Tumor Growth Suppression in Mice: Exponentially growing Lewis lung cancer cells were harvested and washed 3× with saline, and then cell density was adjusted to 10⁷ cells/ml. Cells were then injected 0.5 ml/mouse on the back of each mouse. Mice were grouped into A, B, and C Groups, with 10 mice in each group. Group A were tumor control, Group B were DC-vaccine, and Group C were MEK-DC vaccine mice. Groups A, B, and C were injected with saline, DC vaccine, and MEK-DC vaccine respectively. Vaccinations were performed with 0.5 ml solution once a week. All mice were monitored carefully under constant surveillance. After 6 weeks of vaccination, a tumor in each mouse was removed and weighed. Tumor growth suppression was calculated as follows:

Growth Suppression Rate=(Average Control Tumor Weight−Average Experimental Tumor Weights)/(Average Control Tumor Weights)×100%

None of the mice died after 6 weeks of vaccination. Tumor weights and tumor growth suppression rates are shown in Table 5.

Statistical analyses of tumor weights from each group of mice: P_{AB}<0.05 shows that there was a significant difference between the control group and the group vaccinated with DC: P_{BC}<0.05 shows that the two vaccines resulted in significant differences in retarding tumor growth in mice, where MEK treated vaccine had more effectiveness in tumor growth suppression compared with the DC vaccine.

TABLE 5
Tumor Weights from Mice after 4 Weeks of Vaccination

| Group Rate (%) | Numbers of Mice | Tumor Weight (g) | Suppression |
|---|---|---|---|
| A | 10 | 3.46 ± 0.07 | NA |
| B | 10 | 2.27 ± 0.07 | 34.39 |
| C | 10 | 1.76 ± 0.03 | 49.13% |

### CONCLUSION

The above specific description is meant to exemplify and illustrate the invention and should not be seen as limiting the scope of the invention, which is defined by the literal and equivalent scope of the appended claims. Any patents or publications mentioned in this specification are indicative of levels of those skilled in the art to which the patent or publication pertains as of its date and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material referred to.

What is claimed is:

1. A method of enhancing an immune response to an antigen in a mammal, comprising:
a) isolating dendritic cells from said mammal;
b) contacting said dendritic cells with methionine enkephalin for a time sufficient to stimulate said dendritic cells;
c) contacting said stimulated dendritic cells with said antigen for a time sufficient for said dendritic cells to process said antigen; and
d) injecting the dendritic cells from step c) into said mammal.

2. The method as set forth in claim 1, wherein said antigen is a tumor antigen.

3. The method as set forth in claim 2, wherein said method results in a decrease in size of a tumor in said mammal compared to injecting said dendritic cells not contacted with methionine enkephalin.

4. The method as set forth in claim 1, wherein said dendritic cells are isolated from blood from said mammal.

5. The method as set forth in claim 1, wherein said dendritic cells are isolated from white blood cells.

6. The method as set forth in claim 1, wherein said dendritic cells resulting from step c) and one of (i) methionine enkephalin, (ii) said antigen, or iii) methionine enkephalin and said antigen are injected into said mammal.

7. The method as set forth in claim 1, wherein methionine enkephalin is added at a concentration in the range of about 60 ng/ml to about 120 ng/ml to said dendritic cells.

8. The method as set forth in claim 1, wherein methionine enkephalin is added at a concentration of about 60 ng/ml to said dendritic cells.

9. The method as set forth in claim 1, wherein at least 10⁶ methionine-enkephalin-treated dendritic cells are injected into said mammal.

10. The method as set forth in claim 1, wherein said method results in an increase in activity of cytotoxic T lymphocytes compared to injecting dendritic cells not contacted with methionine enkephalin.

11. The method as set forth in claim 1, further comprising repeating step d) at least one time.

12. A method of stimulating dendritic cells, comprising contacting said dendritic cells with methionine enkephalin.

13. The method as set forth in claim 12, wherein said methionine enkephalin is added at a concentration in the range of about 60 ng/ml to about 120 ng/ml to said dendritic cells.

14. The method as set forth in claim 12, wherein said methionine enkephalin is added at a concentration of about 60 ng/ml to said dendritic cells.

15. The method as set forth in claim 12, wherein said stimulating comprises increasing expression of the cell surface marker CD11_{c} on said dendritic cells.

16. The method as set forth in claim 12, wherein said stimulation comprises increasing secretion of IL-12 from said dendritic cells.

17. The method as set forth in claim 12, wherein said stimulation comprises increasing proliferation of said dendritic cells.

18. A composition for administration to a mammalian subject having a tumor, comprising
a) an antigen associated with said tumor; and
b) dendritic cells stimulated by methionine enkephalin, wherein said cells are obtained from said subject.

19. A method of enhancing an immune response to an antigen in a mammal, comprising administering said antigen and methionine enkephalin into said mammal.

20. The method as set forth in claim 19, wherein said methionine enkephalin is administered intravenously, intra-arterially, intra-muscularly, orally, trans-dermally, parentally, via an inhalation spray, via a nasal drop, via an eye-drop, or via a tablet.

21. The method as set forth in claim 19, wherein said methionine enkephalin is administered prior to, with, or after administration of said antigen.

22. The method as set forth in claim 19, wherein said antigen is a tumor antigen.

23. A composition for administration to a mammalian subject having a tumor, comprising:
a) an antigen associated with said tumor; and
b) methionine enkephalin.
